(19) United States
(12) Patent Application Publication
(10) Pub. No.: US 2005/0049627 A1
(43) Pub. Date: Mar. 3, 2005

(54) Device for controlling bleeding and a method for producing the device

(76) Inventor: Jan Claren, Lund (SE)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 10/492,679

(22) PCT Filed: Oct. 15, 2002
(86) PCT No.: PCT/SE02/01866

(30) Foreign Application Priority Data
Oct. 17, 2001 (SE) 0103454-5

(51) Int. Cl. A61M 29/00

(52) U.S. Cl. 606/193

(57) ABSTRACT

An inflatable uterine device to control uterine bleeding, comprising an inflatable balloon (10) and a hose (11) connecting the balloon to a fluid source. The fluid source comprises an open container (12). A first end of the hose can be integrally connected to the inflatable balloon and a second end of the hose can be integrally connected to the container, and a suspension means (14; 15) is provided at the second end of the hose. A method for producing an inflatable uterine device includes the following steps. Feeding an upper film (18) of plastic material from a first roll (17), feeding a lower film (20) of plastic material from a second roll (19), joining said upper film (18) and said lower film (20) along contour lines forming an inflatable balloon (10), a container (12) and a hose (11) connecting the balloon to the container in an open fluid path, said container (12) having an open end formed by the absence of a joint between said upper film (18) and said lower film (20), and cutting the films outside said contour lines.

The present invention relates to an inflatable uterine device to control uterine bleeding. Uterine bleeding can be caused by post-partum hemorrhages following childbirth. Severe uterine bleedings can be a serious problem due to the large loss of blood. Hemorrhage is one of the major causes of maternal mortality. The invention relates also to a method for producing the uterine device.

### PRIOR ART

A device used for stanching uterine bleeding comprising an expandable balloon and a tubular handle connected therewith for the insertion of the balloon into the uterine cavity is disclosed in U.S. Pat. No. 6,024,753. The device further includes a hose for connecting the balloon to a fluid source. The hose is connected to a pump, which is controlled by a control unit. Both the pump and the control unit will require well-trained personnel and will involve high costs for producing and for operating the device, which is a drawback in several instances.

A similar device is disclosed in U.S. Pat. No. 4,552,557. The pressure of an inflatable portion is measured by a pressure gauge. The device in one embodiment will allow accumulated blood or mucosal debris present in the uterus to be drained.

In the devices disclosed in U.S. Pat. No. 6,024,753 and U.S. Pat. No. 4,552,557 an inflatable balloon is used. The balloon is formed by a material that will allow the balloon to expand and to fill the uterine cavity completely when inflated. The requirements on the material result in a high cost for producing the device. The pump and pressure measuring means will involve further costs and possible problems handling the equipment.

A further drawback is that the devices may include air. Such embedded air has to be removed, so as not to risk that air embolism will occur if the balloon ruptures. The removal of air involves further costs.

### SUMMARY OF THE INVENTION

It is an object of the present invention to provide a device for controlling or stanching uterine bleedings that is without the drawbacks mentioned above. The device according to the invention can be produced at a very low cost and can be used under very primitive conditions. No pump or similar device is necessary to provide the required pressure for inflating the balloon and for exerting sufficient pressure on uterine blood vessels to curtail bleeding.

According to the invention the fluid source comprises a container with an opening that will allow filling of a fluid, preferably water, into the container and through the hose into the balloon. Preferably, the opening is formed as a funnel, so as to facilitate the filling of fluid.

The hose close to the container or the container itself is provided with suspension means. The suspension means can be used to suspend the device at such a height in relation to the balloon that the pressure in the balloon will reach a sufficient level. Normally, the surface of fluid in the container will be kept at least 1 m above the balloon. A 1 m long hose corresponds to a pressure in the balloon of about 70 mm Hg, which in most circumstances will be sufficient.

The length of the hose will normally be no more than 3 meters, thus restricting the maximum pressure that can be obtained from the water pressure to about 220 mm Hg (29.3 kPa). At this pressure neither the tissue in the uterine cavity nor the uterus itself can be damaged. A working pressure of approximately 70 mm Hg would be a normal pressure. The hose may be provided with markings over a section or the complete length. The markings indicate the pressure in the balloon when the container is lifted. The hose may also be provided with a more detailed scale that can be used if a pressure clamp is used to raise the pressure in the system.

The device, comprising the container, the hose and the balloon, according to the present invention can all be in an integral form and no connectors or similar devices are required to be present. When inflated the balloon will fill the uterine cavity and exert a sufficient pressure on the surrounding tissue to curtail bleeding.

Different methods can be used for producing the device according to the invention. In one embodiment two sheets that are welded together in the appropriate shape form the device. The sheets are fed from two rolls to a welding and cutting device. Preferably, side edges of the device are welded and cut in one step. The cut out device is then either folded to an appropriate shape and size or rolled into a roll. The device is sterilised and wrapped in a package. If folded the device preferably is disposed on a sheet or plate that will function as a base plate when the device is unpacked. As a result of this producing method no air will be present within the device. As no cavities with air will be present in the device it is ensured that no air embolism occurs during use of the device.

The package may include a clamp or similar device that can be used to close the container and/or to raise the pressure in the balloon. In the latter case the clamp can be moved over the hose from the container towards the balloon and may include rolls or other rotating means.

The balloon has a size when inflated that is sufficient to fill the uterus of most women. Preferably a non-resilient material is used. A major advantage of using a non-resilient material is that the pressure in the area of filling the device, a filling pressure, will equal to a working pressure of the device. Thus, there will be no need for measuring the pressure of the balloon when it is located in the uterus.

The term non-resilient should be understood to include materials that are completely without resilience and materials that will have a low degree of resilience and a substantially linear relation between expansion and pressure increase. When a non-resilient material is used the volume of the inflated balloon is at least 0.3 litre. In prior art devices the resilient material will cause a resistance when the pressure is increased. Therefore, the pressure normally has to be measured within the uterus.

It is possible to produce the device at very low costs. Biocompatible but low-cost materials can be used, because it is not required that the material is resilient. Large amounts of the device can be distributed in developing countries to save many lives.

### BRIEF DESCRIPTION OF THE DRAWINGS

FIG. 1 is a schematic plan view of the device according to the invention.

FIG. 2 is a schematic side elevation view of a production line for producing the device according to the invention.

FIG. 3 is a schematic plan view of the production line in FIG. 2.

FIG. 4 is a cross sectional view from line IV-IV in FIG. 1.

FIG. 5 is a schematic plan view of the device in FIG. 1 on a base plate with folding lines indicated with dashed lines.

FIG. 6 is a schematic plan view of the device in FIG. 5 in a folded condition.

FIG. 7 is a schematic side elevation view of the folded device.

FIG. 8 is a schematic side elevation view of the device rolled into a product roll.

FIG. 9 is a schematic side elevation view of the device in FIG. 1 extended and filled with liquid and an enlarged section showing a clamping element.

FIG. 10 shows schematically a first embodiment of an introducing member.

FIG. 11 shows schematically a second embodiment of an introducing member.

### DETAILED DESCRIPTION

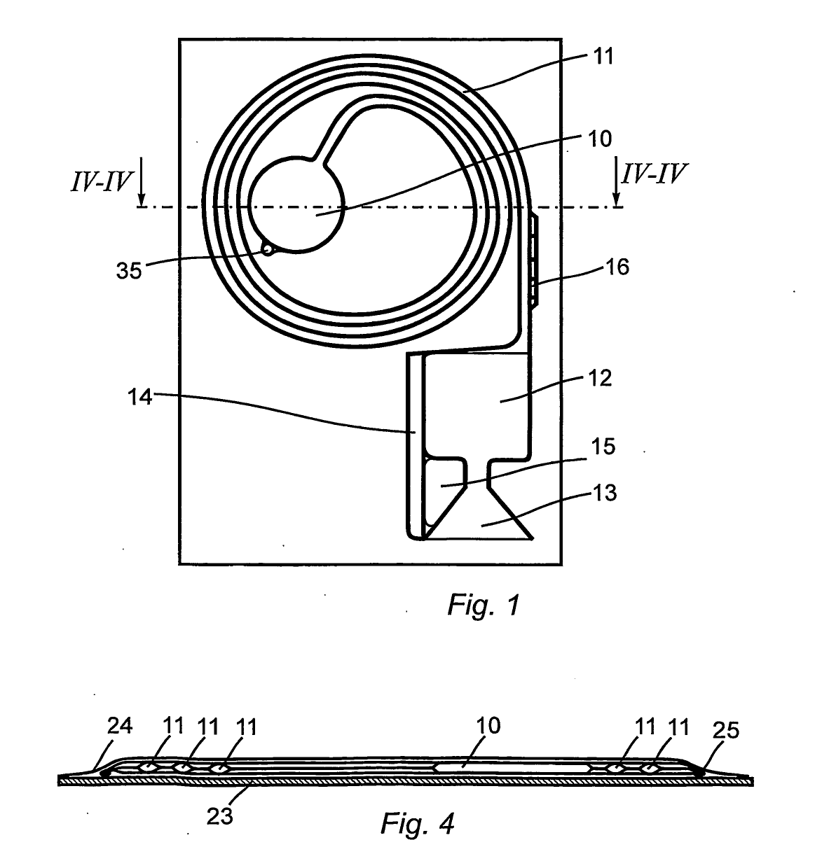

Referring to FIG. 1, it can be seen that a device according to the invention comprises three main sections. It should also be noted that the device is shown in a configuration when delivered and ready for use. A balloon 10 constitutes the part of the device that is designed to fit in the human uterus. The balloon 10 is circular or approximately circular and completely flat in a non-inflated condition. In an inflated condition (not shown) it will assume a somewhat flattened but spherical shape. The volume of the balloon 10 in the inflated condition is between 0.3 litre and 2.5 litres, corresponding to a diameter of about 0.13 m to about 0.20 m. An appropriate size is achieved with a diameter of 0.155 m resulting in a maximum volume of about 1.5 litre. A balloon of that size will for most women exert a sufficient pressure, even if the balloon is not fully inflated. As an alternative two sizes of the balloon are provided. A smaller sized balloon will hold from about 0.3 l to about 1.5 l, and a larger sized balloon will hold from about 1 l to about 2.5 l.

A balloon that is not fully inflated will adapt to the form of the uterus more closely and the shape of the balloon is of less importance. By using thin films the balloon can fill small and irregularly shaped cavities. Other sizes of the balloon may be provided for specific conditions such as when a differently sized uterus is to be treated. The design of the device and the thin material used will allow the use of more than one balloon if necessary.

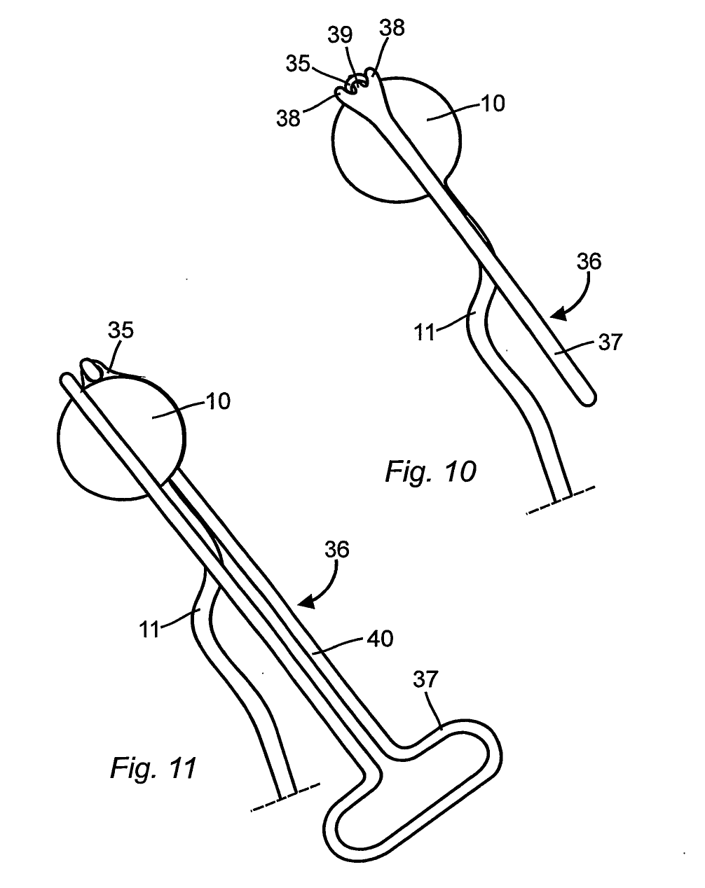

An annular flap 35 can be provided on the edge of the balloon opposite to the connection between the hose and the balloon. The flap 35 can receive a tip of an introducing member, for instance as the one shown in FIG. 10.

The balloon is integral with a hose 11 extending from the balloon to a container 12. The length of the hose 11 from a first end connected to the balloon to a second end connected to the container 12 will be sufficient to give the required pressure when the container 12 is filled with water and raised. The container is designed to be filled with a liquid, such as water, and has one open end where water can be filled and an opposite end, which is integrally connected to the hose. In the embodiment shown in FIG. 1 the open end is shaped as a funnel 13, so as to facilitate the filling of water. The funnel shaped opening 13 will allow water to be poured into the container.

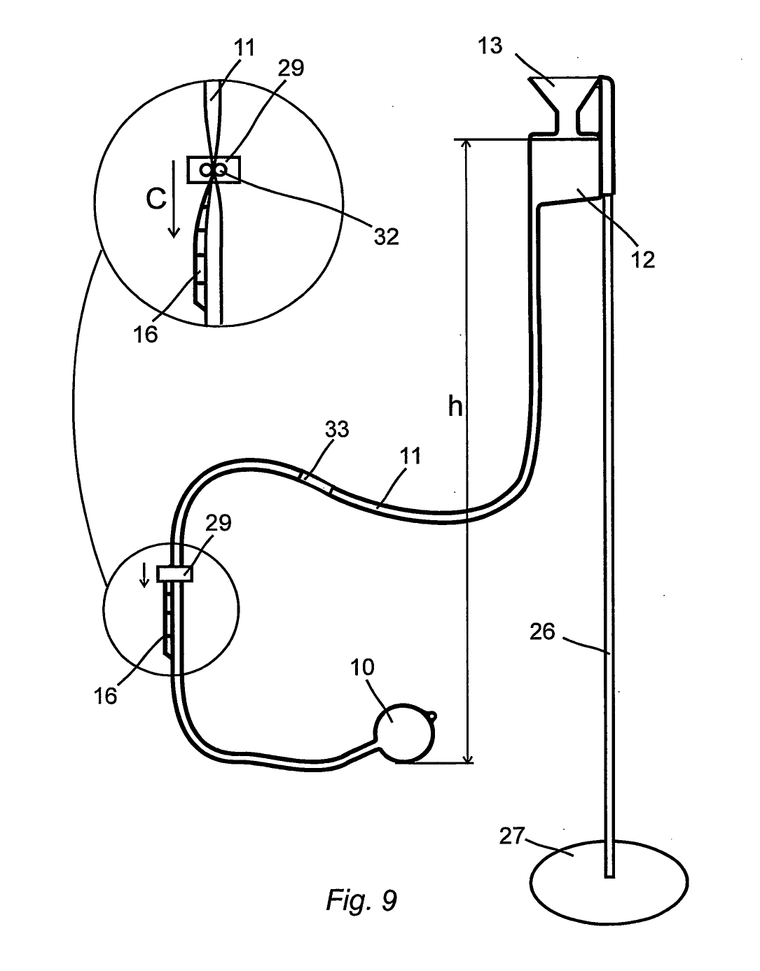

The container 12 is provided with a suspension means to facilitate the suspension thereof in a raised position. In the embodiment shown in FIG. 1 a longitudinal pocket 14 extending from a lower part of the container 12 to the funnel shaped opening 13 is used. The pocket is formed to receive a vertical rod or pin and is open in one end and closed in the other end. The open end is located at the lower part of the container 12. FIG. 9 shows an example of the use of the suspension means. Also a recess 15 formed between the pocket 14 and the funnel shaped opening 13 can be used as a suspension means.

At an appropriate distance from the balloon 10 the hose 11 is provided with a scale 16 that can be used when an increased pressure is required. The use of the scale is further described with reference to FIG. 9 and the enlarged section.

In a preferred embodiment two sheets that are welded together form the device. A space between the sheets formed by welding seams can be filled with a liquid such as water as disclosed above. The complete device is mounted on a plate or sheet 23 that will hold the device and facilitate the handling thereof when the device is to be used. The plate 23 can be made from paper only or from a plastic film coated paper sheet.

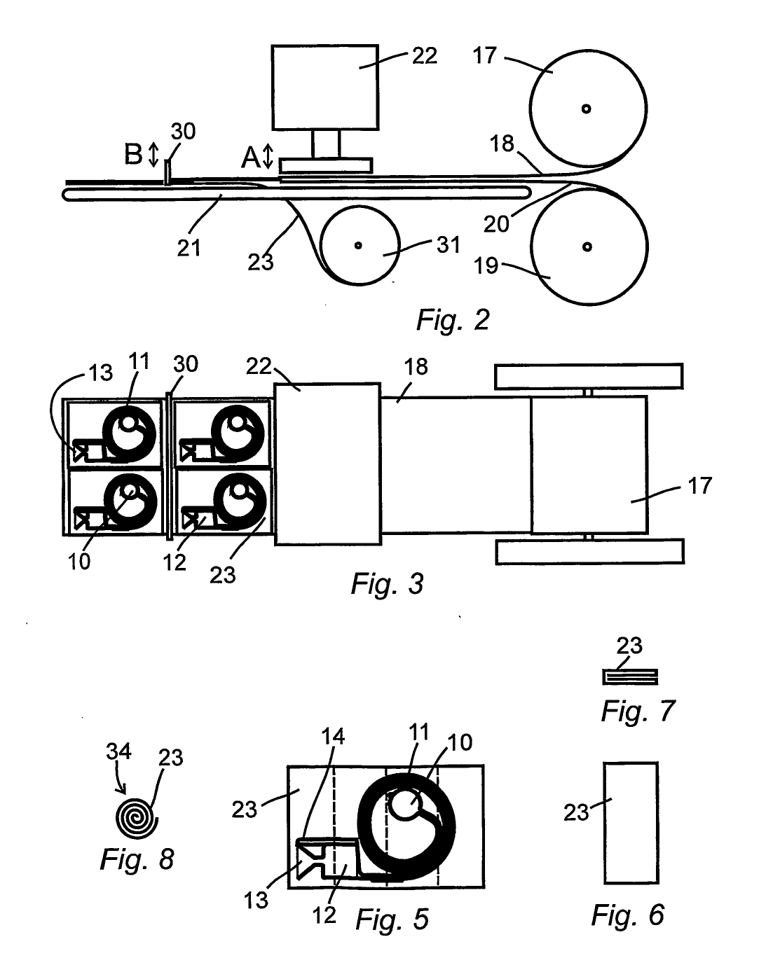

An example of a production line for the manufacturing thereof is shown in FIG. 2. A plastic film is rolled on a first roll 17 and forms an upper film 18. A corresponding plastic film is rolled on a second roll 19 and forms a lower film 20. The plastic film can be made from different plastic materials with appropriate properties. The material should allow a combined cutting and welding process and should also be biocompatible. Suitable materials are polypropylene, polymers such as ethylene methacrylic acid copolymer (EMA) and Ethylene Vinyl Acetate (EVA). At least the balloon is formed by thin films of a rigid or non-flexible material that are welded together.

The upper film 18 and the lower film 20 are conveyed over a working table 21 to a welding and cutting device 22. The films are welded together along the contour lines forming the device as shown in FIG. 1. There are no welded seams in the funnel shaped opening 13 and in the opening of the pocket 14. In a preferred embodiment the seams are welded and the device is cut out at the same time.

The welding and cutting device 22 can include a press form or a similar device that moves up and down as shown at arrow A, or a laser that welds and cuts in one step. Also other welding techniques, such as RF-heat-welding can be used.

As can be seen in FIG. 3 the films 18, 20 are so wide that two devices fit beside each other. The films can be even wider, so as to allow three or more devices to be welded and cut at the same time.

In connection with or after the welding and cutting steps the device is mounted on the plate or sheet 23, which can be formed by paper or a plastic material. The sheet 23 is fed from a third roll 31 below the working table 21. In a preferred embodiment the device is attached to the plate 23 in the welding seams along the contour of the device. It is then a simple measure to release the device section by section from the plate 23 when the device is used.

In an alternative embodiment the sheet 23 engages the two films before the welding and cutting device 22. Thereby the films will adhere to the sheet 23 during the cutting and welding step. The complete device including the plate 23 is then wrapped into a package. A cutting device 30 moving up and down as shown at arrow B in FIG. 2 cuts the plates into suitable sizes. The cutting device can also be a rotating device or a laser device or any other appropriate device.

The device is sterilised with an appropriate method. Among the sterilization technologies currently available, ethylene oxide (EtO) gas can be mentioned. It is also possible to use other methods, such as gamma radiation and electron-beam radiation. Normally, the device is sterilised in the package.

FIG. 4 is a cross sectional view of the complete device including the plate 23 wrapped in a package. It should be noted that the extension of the device in the vertical direction is exaggerated to facilitate the understanding of the invention. A plastic covering film 24 is wrapped over the device and attached to the plate 23 to form a sealed and sterile package. Three sections of the hose 11 and the balloon 10 are discernible as separate hollow spaces. These hollow spaces will be inflated when a liquid is poured into the container 12. Welded seams 24 are formed where the device is attached to the plate 23.

Dashed lines in FIG. 5 indicate folding lines where the device can be folded to assume the shape shown in FIG. 6 and FIG. 7. The device is folded twice towards the centre of the device and the plate 23 will form a protective cover. By folding the device as shown an extremely compact package will be achieved. The size can be as small as a few millimetres thick, 350 mm long and 185 mm wide. A package of that size can be stored and distributed at very low costs, which in most cases, such as for use in developing countries, is advantageous.

It is also possible to roll the device to form a product roll. FIG. 8 shows a product roll 34 where the sheet 23 forms a protective exterior of the roll.

FIG. 9 illustrates schematically the device in use. It should be assumed that the balloon is located within the uterus. The container 12 is suspended on a vertical rod 26 extending from a base plate 27. The longitudinal pocket 14 is used for suspending the device. A liquid such as water has been poured into the container through the funnel shaped opening. An assumed water level is indicated at 28 and is located at a height of h above the balloon. As discussed above h should be in the interval of 1-3 m. The length of the hose is from about 1.0 m to about 3.0 m and preferably from about 1.5 m to 2.5 m. A suitable lower distance h=1.0 m corresponds to a pressure of about 70 mm Hg. If air may enter the space between the films during or after the manufacturing of the device a priming step is taken before the device is used.

If it is desirable to increase the pressure in the balloon further a clamping element 29 can be mounted on the hose 11, preferably in the area of the scale 16. The clamping element 29 comprises in the shown embodiment two cylinders or wheels 32 bearing against each other. The cylinders 32 can be moved apart, so as to allow the hose to be inserted there between. The cylinders 32 are then pressed against each other as shown in the enlarged detail view in FIG. 9. In another embodiment (not shown) one cylinder rotates against an abutment.

By moving the clamping element 29 in the direction of arrow C liquid within the hose will be forced toward the balloon where the pressure will rise. The increased pressure within the balloon will contribute to provide a sufficient pressure on uterine blood vessels to curtail bleeding. The position of the clamp 29 in relation to the scale will indicate the pressure in lower part of the hose and in the balloon.

The clamping element 29 can also be used to close the container. It is then possible to increase the working pressure by enclosing the container 12 in a cuff or similar device. The pressure of the cuff is increased in a conventional manner and will lead to a corresponding increase of the working pressure. An advantage of using a non-resilient material is that the pressure that can be observed in the cuff will be equal to the working pressure of the balloon and the complete device.

FIG. 10 shows an introducing member 36 that can be used to introduce the balloon into the uterus. In the embodiment shown the introducing member comprises an elongated handle 37. The handle is in one end provided with a head having two extending rounded sections 38 and between said sections a rounded tip 39. The rounded tip has a size that will allow it to be inserted in the annular flap 35, when the balloon is to be introduced into the uterus. The two side sections 38 extend beyond the rounded tip and will ensure that no damage is caused to the uterus.

The introducing member can be rigid and be made from a plastic material. It is also possible to form it in paper from the sheet 23. In that case the introducing member is punched. In another embodiment the introducing member is formed by a gel that will dissolve in contact with blood and other body fluids.

In the alternative embodiment shown in FIG. 11 the introducing member 36 comprises a handle 37 with two sticks 40 extending in parallel from the handle. One of the sticks is arranged on one side of the flat balloon and then inserted through the annular flap 35 while the other stick extends on the opposite side of the balloon. With this arrangement it is possible to wind the empty flattened balloon around the introducing member and then to introduce it in the uterus.

Normally, the balloon is introduced into the uterus empty and then a smaller amount of liquid is filled into the balloon. When the balloon is inflated by the liquid the introducing member can be withdrawn leaving the balloon in the uterus. Then a suitable amount of liquid is filled into the container 12 and the container is raised to an appropriate height over the balloon.

In one embodiment the material and the thickness of the hose are chosen so as to withstand a pressure lower than a pressure where any tissue is damaged. When the clamping element is used and the pressure is increased over what can be obtained by positioning the container at a maximum distance from the balloon the limited strength of the material will ensure that the pressure is not increased to an injurious level. At higher pressures the hose, or any other part of the device exposed to the pressure, will rupture. It may be appropriate to provide the hose with a specific rupture section 33 that will be an indication of fracture. The section can be formed with a weakened material or as a thinner part.

In an embodiment where all elements of the device are integrally connected to each other the device can be produced, stored and transported very efficiently. The storage and transport will be simplified also as a result of the thin and compact design. No separate parts or connectors have to be used, unless a clamping device is included. This design will facilitate in maintaining the device under sterile condition also during troublesome circumstances.

1. An inflatable uterine device to control uterine bleeding, comprising an inflatable balloon (10) and a hose (11) connecting the balloon to a fluid source, characterised in
that the fluid source comprises a fillable container (12), and
that the inflatable balloon (10) is made from a substantially non-resilient material.

2. An inflatable device as claimed in claim 1, wherein a suspension means (14; 15) is provided at the second end of the hose.

3. An inflatable device as claimed in claim 1, wherein a first end of the hose is integrally connected to the inflatable balloon and a second end of the hose is integrally connected to the container.

4. An inflatable device as claimed in claim 1, wherein an opening (13) of the container (12) is provided to allow filling of liquid into the container.

5. An inflatable device as claimed in claim 4, wherein said opening (13) is funnel shaped.

6. An inflatable device as claimed in claim 1, wherein the length of the hose (11) is sufficient to allow the container (12) to be elevated a distance from the balloon (10) corresponding to a water column that exceeds 1 m.

7. An inflatable device as claimed in claim 1, wherein the balloon (10) has a volume ranging from 0.3 l to 1.5 l.

8. An inflatable device as claimed in claim 1, wherein the balloon (10) has a volume ranging from 1 l to 2.5 l.

9. An inflatable device as claimed in claim 1, wherein the balloon (10), the hose (11) and the container (12) all are formed by the same material.

10. An inflatable device as claimed in claim 1, wherein a rupture section (33) is formed in the hose (11).

11. An inflatable device as claimed in claim 1, also comprising a clamping element (29), said clamping element (29) comprising at least one rotating means pressing a section of the hose and closing the hose.

12. A method for producing an inflatable uterine device to control uterine bleeding, characterised by
feeding an upper film (18) of plastic material from a first roll (17),
feeding a lower film (20) of plastic material from a second roll (19),
joining said upper film (18) and said lower film (20) along contour lines forming the device and
cutting the films outside said contour lines.

13. A method as claimed in claim 12, wherein the contour lines forms an inflatable balloon (10), a container (12) and a hose (11) connecting the balloon to the container in an open fluid path, said container (12) having an open end formed by the absence of a joint between said upper film (18) and said lower film (20).

14. A method as claimed in claim 12, also including the step of joining said upper film (18) and said lower film (20) simultaneously with said cutting.

15. A method as claimed in claim 12, also including the steps of feeding a sheet (23) from a third roll (31) below said lower film (20), adhering the joined upper film (18) and lower film (20) to said sheet (23) and cutting the sheet (23) to form a plate including said inflatable balloon (10), said container (12) and said hose (11).

16. A method as claimed in claim 15, also including the steps of folding twice opposite side edges of said plate towards each other and enclosing said upper film (18) and said lower film (20) between sides of said sheet (23).

17. A method as claimed in claim 15, also including the step of rolling the plate into a product roll (34), the sheet (23) forming the outside of the product roll.

18. A method as claimed in claim 12, also including the step of feeding a sheet (23) from a third roll (31) below said lower film (20), joining said upper film (18) and said lower film (20) and adhering the films to the sheet (23) simultaneously.

19. A method for controlling uterine bleeding, characterised by introducing an inflatable non-resilient balloon in the uterus,
filling a liquid into a fillable container that is connected to the balloon through a hose,
suspending the container at a distance above the balloon corresponding to a pressure in the balloon sufficient to end the uterine bleeding.

20. A method as claimed in claim 19, including the step of suspending the container at least one meter above the balloon.

21. A method as claimed in claim 19, including the steps of connecting a an elongated introducing member to the balloon and inserting at least a tip of the introducing member into the uterus bringing the balloon to a position inside the uterus.
